(19) United States
(12) Patent Application Publication
(10) Pub. No.: US 2004/0192553 A1
(43) Pub. Date: Sep. 30, 2004

(54) Inosine L-Arginine salt and uses thereof

(76) Inventors: Masahiko Kurauchi, Kanagawa (JP); Yuki Miyazawa, Kanagawa (JP); Hiroyuki Sato, Kanagawa (JP)

Correspondence Address: AJINOMOTO CORPORATE SERVICES, LLC, INTELLECTUAL PROPERTY DEPARTMENT, 1120 CONNECTICUT AVE., N.W., WASHINGTON, DC, 20036, US

(21) Appl. No.: 10/808,536

(22) Filed: Mar. 25, 2004

Related U.S. Application Data
(63) Continuation of application No. PCT/JP02/09184, filed on Sep. 10, 2002.

(30) Foreign Application Priority Data
Sep. 27, 2001 (JP) 2001-297011

(51) Int. Cl. A61K 31/715; A01N 43/16

(52) U.S. Cl. 504/292; 514/54; 536/55.1; 514/46

(57) ABSTRACT

The present invention discloses an inosine. L-arginine salt, compositions containing the salt, and methods of using the salt and said compositions for cell activation and/or plant growth promotion. The salt can be stored and transported as a solid and dissolves quickly and efficiently when needed.

This application is a continuation of PCT/JP02/09184, filed Sep. 10, 2002.

### BACKGROUND OF THE INVENTION

1. Field of the Invention

The present invention relates to an inosine. L-arginine salt and compositions containing this salt. The present invention also relates to methods of activating cells and promoting plant growth using the salt, or compositions thereof.

2. Brief Description of the Related Art

Inosine is known to have cell activiation activity. Therefore, due to its known medicinal benefit, products containing inosine have been marketed for treatment of leukopenia, which is caused by exposure to radiation or certain drugs. These products include Inothine Injection Solution (Towa Pharmaceutical CO.,LTD.) and Inosine Granule (MARUKO Pharmaceutical CO., LTD.). Furthermore, a complex salt of inosine with 4-acetamidobenzoate 2-hydroxypropyldimethylammonium has been marketed by MOCHIDA PHARMACEUTICAL CO., LTD. as Isoprinosine Tablet, with the claim that it has a positive effect for patients of subacute sclerosing panencephalitis, such as prolonging survival time.

Furthermore, the effect of inosine on promoting plant growth is also known. For example, Japanese Patent No. 2927269 discloses a growth promoter for plant roots which contains inosine as an active ingredient. This growth promoter is marketed by Shoko-tsusho CO., Ltd. under the Japanese name IKUOU. In addition, Japanese Patent Application Laid-open (Kokai) No. 2001-72514, by the same inventor, includes an example using inosine calcium salt or free inosine.

The solubility of inosine in water is relatively low. Specifically, inosine solubility is only about 1.6% at normal temperatures. Therefore, when the use of inosine in a solution is necessary, the dissolution time period is prolonged and the conentration is limited. Japanese Patent No. 2927269, mentioned above, describes this problem and the process of making a strong alkaline solution of inosine using caustic alkali and the like to solve the problem. However, these methods generate a safety hazard, as is described in the above-mentioned Japanese Patent Application Laid-open (Kokai) No. 2001-72514, which is by the same inventor. In addition, the above-described solutions are not stable when stored, and furthermore, transport of the above-described solutions is costly.

Accordingly, there is a need in the art to to develop an inosine formulation that can be distributed as a solid and dissolved quickly when used, eliminating the need for stable storage environments and expensive transport of the solutions. The present invention solves this problem and discloses a stable inosine. L-arginine salt, which dissolves easily in water while retaining its plant growth promoting and cell activating properties.

### SUMMARY OF THE INVENTION

It is an object of the present invention to provide an inosine. L-arginine salt.

It is a further object of the present invention to provide the inosine. L-arginine salt described above, wherein inosine and L-arginine are present in substantially equimolar amounts.

It is a further object of the present invention to provide the inosine. L-arginine salt described above produced by the process of dissolving in water inosine and L-arginine in substantially equimolar amounts.

It is an even further object of the present invention to provide the inosine. L-arginine salt described above comprising the additional step of adding the dissolution product to anhydrous ethanol.

It is an even further object of the present invention to provide the inosine. L-arginine salt described above comprising the additional step of drying the dissolution product.

It is a further object of the present invention to provide a composition comprising an inosine. L-arginine salt.

It is a further object of the present invention to provide a composition comprising an aqueous solution of inosine and L-arginine.

It is a further object of the present invention to provide the composition described above, wherein said inosine and said L-arginine are present in substantially equimolar amounts.

It is a further object of the present invention to provide a method of promoting the growth of a plant comprising treating said plant with an inosine. L-arginine salt or a composition comprising an aqueous solution of inosine and L-arginine, wherein said inosine and said L-arginine are present in substantially equimolar amounts.

It is a further object of the present invention to provide a method of activating a cell comprising treating the cell with an inosine. L-arginine salt or a composition comprising an aqueous solution of inosine and L-arginine, wherein said inosine and said L-arginine are present in substantially equimolar amounts.

It is a further object of the present invention to provide a method making an inosine. L-arginine salt comprising (a) dissolving in water inosine and L-arginine in substantially equimolar amounts; (b) adding the product of step (a) to anhydrous ethanol; and (c) drying the product of step (b) to obtain inosine. L-arginine salt.

Still other objects, features, and attendant advantages of the present invention will become apparent to those skilled in the art from a reading of the following detailed description of embodiments constructed in accordance therewith, taken in conjunction with the accompanying drawings.

### BRIEF DESCRIPTION OF THE DRAWINGS

The invention of the present application will now be described in more detail with reference to preferred embodiments of the product and methods, given only by way of example, and with reference to the accompanying drawings, in which:

FIG. 1 shows the infrared spectroscopic analysis of a mixture of inosine and L-arginine in equimolar amounts.

FIG. 2 shows the infrared spectroscopic analysis of the inosine. L-arginine salt prepared in Example 1.

FIG. 3 shows the powder X-ray diffraction pattern of the inosine. L-arginine salt prepared in Example 1.

FIG. 4 shows the powder X-ray diffraction pattern of a mixture of inosine and L-arginine in equimolar amounts.

### DESCRIPTION OF THE PREFERRED EMBODIMENTS

As a result of their intensive studies to solve the above-described problems, the present inventors have found that when inosine is made into an arginine salt, it can be isolated as a solid which is capable of dissolving very easily in water. Accordingly, the present invention first relates to the salt of inosine with L-arginine, the inosine. L-arginine salt. This salt can be isolated as a solid and is capable of being easily dissolved in water. Furthermore, this salt, or a composition containing the salt, is useful in methods of activating cells and promoting plant growth. The present invention also relates to a composition comprising an aqueous solution of inosine and L-arginine

The above-mentioned Japanese Patent No. 2927269 discloses basic amino acids such as lysine, arginine and the like, as the alkaline agent in addition to caustic alkali agents. However, the purpose of the alkaline agent is merely to permit the poorly soluble inosine to be in solution. There are no reports on a method for isolating the salt of inosine with a basic amino acid as a solid. Further, there are no reports on inosine. L-arginine salt.

According to the studies of the inventors, when inosine is neutralized and dissolved with equimolar amount of arginine in water, followed by a simple concentratration, the solid separated out is pure inosine. In order to obtain inosine. L-arginine salt as a solid, a concentrated solution containing inosine and L-arginine in equimolar amounts is added to anhydrous ethanol while stirring. The concentration of this concentrated solution can be any amount as chosen by one of skill in the art. For example, at 60 ° C., the concentration of the solution will be 50% or more. Inosine. L-arginine salt is represented by the molecular formula C₁₆H₂₆N₈O₇ and is a novel substance which has never been described in the literature. Inosine. L-arginine salt may contain water and/or a solvent (hydrate and/or solvate) depending on the preparation conditions. Inosine. L-arginine salt may also be obtained, for example, by freeze-drying or spray-drying an aqueous solution containing arginine in an amount equimolar with inosine.

An aqueous solution of inosine and L-arginine wherein the inosine and the L-arginine are present in substantially equimolar amounts, has never been described in the literature either, and is therefore a novel composition. Such an aqueous solution may be prepared, for example, by weighing out inosine and L-arginine in equimolar amounts, respectively, and adding them to water while stirring.

Here, the phrase “substantially equimolar amounts” means that inosine and L-arginine are present in equal molar amounts, but these amounts can vary slightly. That is, the inosine. L-arginine salt may be dissolved in an aqueous solution containing previously dissolved inosine and/or L-arginine. Alternatively, inosine or L-arginine may be dissolved later in an aqueous solution in which the inosine. L-arginine salt has been dissolved. Concentrations of such mixed aqueous solutions are not particularly limited. For ease and efficiency of transporting the aqueous solution and the like, higher concentrations are more preferable. A highly concentrated solution can be suitably diluted for actual use.

A mixed aqueous solution of inosine and L-arginine can of course be prepared by dissolving inosine. L-arginine salt in water. In this case, the inosine and the L-arginine are present in an equimolar ratio in the resulting aqueous solution, in accordance with the fact that inosine. L-arginine salt consists of equimolar amounts of inosine and L-arginine.

Furthermore, a solution of inosine and L-arginine can be also obtained by dissolving inosine. L-arginine salt in water which already contains dissolved inosine and/or L-arginine. In the mixed aqueous solution of inosine and L-arginine thus obtained, a solute such as inosine, L-arginine or the like may be further dissolved. Concentrations of such mixed aqueous solutions are not particularly limited. For ease and efficiency of transporting the aqueous solution and the like, higher concentrations are more preferable. Again, a highly concentrated solution can be suitably diluted for actual use.

The inosine. L-arginine salt may be used as an active ingredient in a pharmaceutical composition for treatment of leukopenia and the like. Such compositions may contain a vehicle, lubricant, flavoring substance, or the like, which are ordinarily used when upon preparing formulations for pharmaceutical use. The ratios and amounts of these ingredients are well known to those of ordinary skill in the art.

The composition containing inosine and L-arginine may also be in the form of a liquid formulation, such as an aqueous solution. The composition having inosine and L-arginine as active ingredients may be prepared by the process of dissolving the inosine and the L-arginine in water in substantially equimolar amounts. In these cases, the concentrations can vary as long as the solutes are capable of being dissolved. Preferably, the concentrations are about 5% at 25° C.

The phrase “treating the cell” includes all forms administering a solid or liquid composition to a cell or a group of cells, and can be accomplished by conventional methods by those with ordinary skill in the art.

The inosine. L-arginine salt, or compositions containing the inosine. L-arginine salt as an active ingredient, or compositions comprising an aqueous solution of inosine and L-arginine in equimolar amounts may be used in methods for promoting the growth of above-ground and/or under-ground parts of beans such as soybean, red bean, and the like; potatoes such as Irish potato, sweet potato, and the like; grains in general, including poaceous plants such as barley, wheat, rice plant, sweet corn and the like; culinary vegetables in general, including leaf vegetables such as lettuce, cabbage, spinach, and the like, root vegetables such as radish, carrot, and the like; and fruit vegetables such as tomato, cucumber, green pepper, eggplant, pumpkin, and the like; fruits in general, including fruit trees such as apple, pear, grape, cherry fruit, peach, citrus, and the like, and fruit vegetables such as strawberry, melon and the like; and other useful plants in general, excluding edible ones, such as cotton plant, tobacco and the like.

The phrase “treating the plant” means that the inosine. L-arginine salt, or compositions containing the inosine. L-arginine salt as an active ingredient, or compositions comprising an aqueous solution of inosine and L-arginine in equimolar amounts is applied to the plant by conventional means known to those of ordinary skill in the art depending on the physical form of the inosine. L-arginine salt or the compositions. For example, if in solid form, the salt may be sprinkled on of over the plant, or buried in the soil surrounding the underground parts of the plant. If in liquid form, for example, the compositions of the present invention may be sprayed onto the plants and/or the soil. The inosine. L-arginine salt composition may include salt(s) of iron, copper, manganese, zinc, molybdenum, boron, cobalt, potassium, calcium, magnesium or the like in addition to inosine. L-arginine salt. It may also contain amino acid(s) such as proline, serine, glutamine or the like. The ratios and amounts of these ingredients are well known to those of ordinary skill in the art. Furthermore, the compositions of the present invention which are useful as a plant growth promoter may also be in the form of a liquid formulation, such as an aqueous solution. The composition having inosine and L-arginine as active ingredients may be prepared by the process of dissolving the inosine and the L-arginine in water in substantially equimolar amounts. In these cases, the concentrations can vary as long as the solutes are capable of being dissolved. Preferably, the concentrations are about 5% at 25 ° C.

### EXAMPLES

Hereinafter, the present invention will be described further in detail with reference to non-limiting examples.

### Example 1

Preparation of an Inosine. L-arginine Salt

20.00 g (74.6 mmol) of inosine and 13.00 g (74.6 mmol) of L-arginine were added to 67 mL of water. The resulting mass was warmed and shaken in a hot-water bath until the solutes were completely dissolved. The resulting aqueous solution was added little by little to 2 L of ethanol while vigorously stirring, resulting in a white solid precipitated. The solid was filtered and collected. The weight of the solid was measured at 25.65 g after drying overnight in a reduced pressure at 40° C.

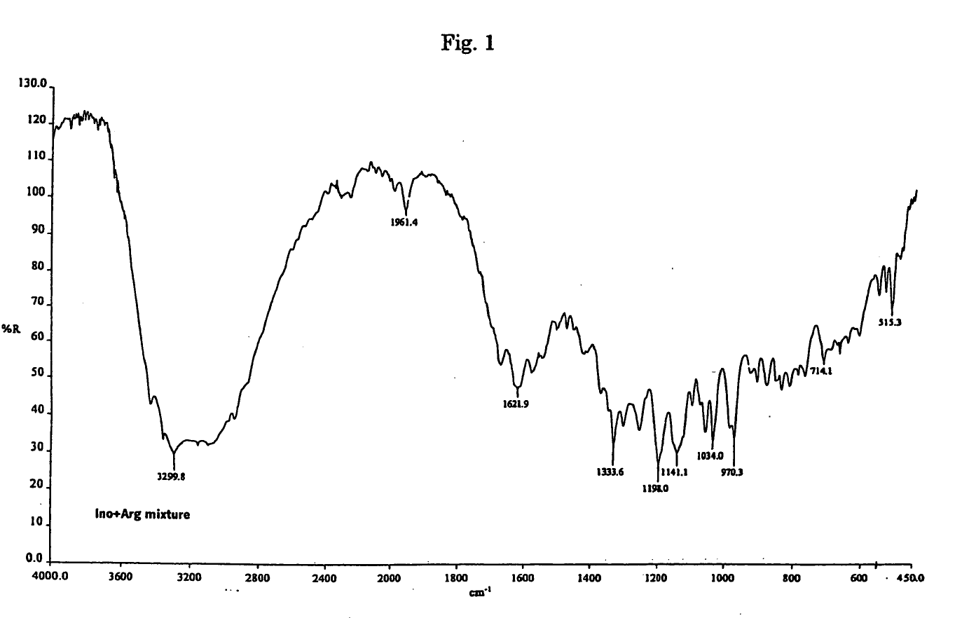

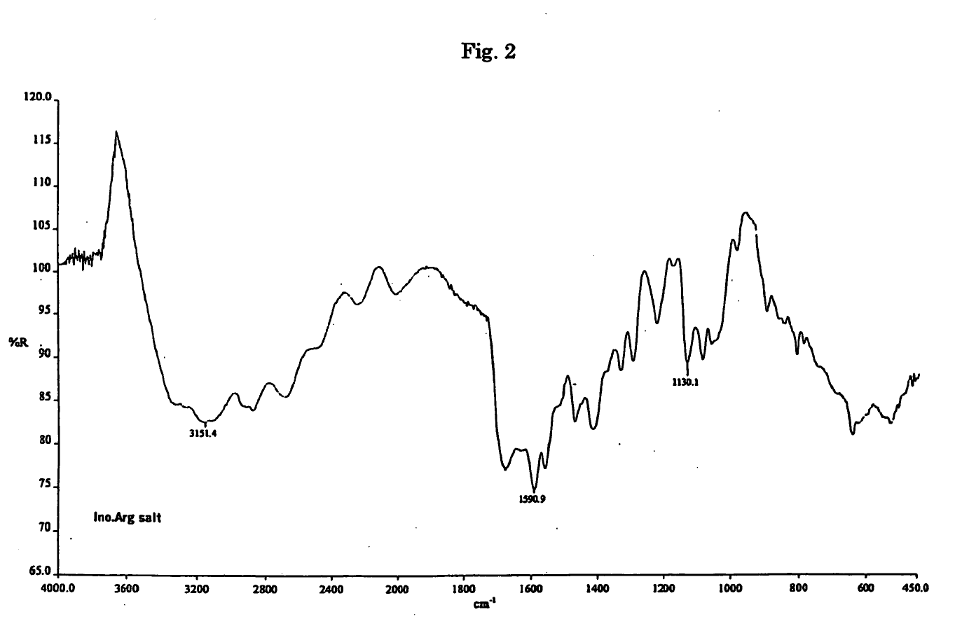

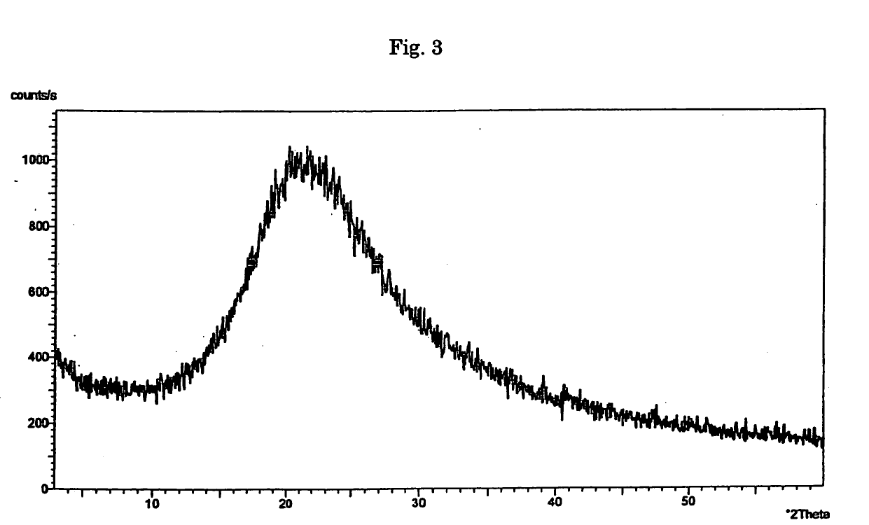

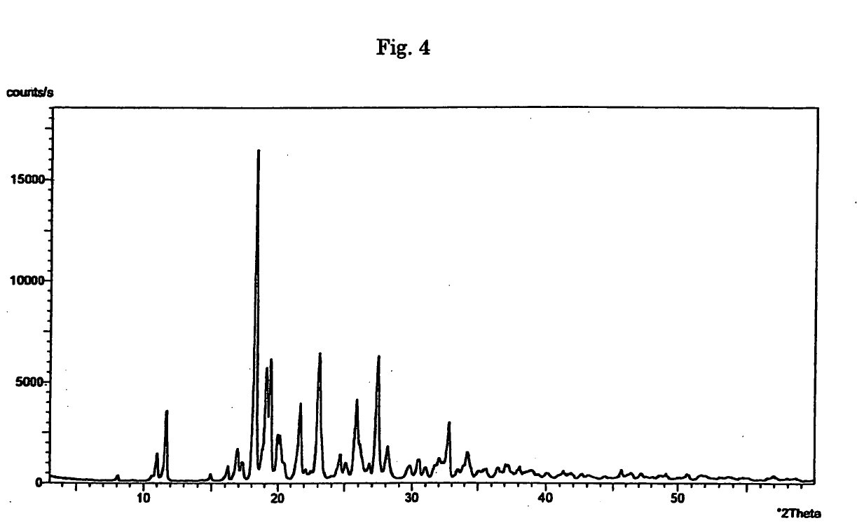

The solid was analyzed with a high speed liquid chromatography apparatus. Inosine and L-arginine having a molar ratio of 1:1 were detected. The mixture was compared to an equimolar mixture of inosine and L-arginine by infrared spectroscopic analysis (see FIG. 1). It was shown that the peak at 1961 cm⁻¹, which is characteristic of the mixture, had disappeared (see FIG. 2). As the result of powder X-ray diffraction, the solid was shown to be amorphous (FIG. 3). Elemental analysis showed the following: C:43.57%, H:6.43% and N:23.24%. This coincides with the theoretical value of inosine. L-arginine salt. 0.6 ethanol. 0.4 hydrate (C:43.28%, H:6.42% and N:23.48%). Furthermore, powder X-ray diffraction of an equimolar mixture of inosine and L-arginine was performed to obtain a chart for comparison (see FIG. 4).

### Comparative Example 1

20.00 g (74.6 mmol) of inosine and 13.00 g (74.6 mmol) of L-arginine were added to 400 mL of water. The resulting mass was stirred at room temperature to completely dissolve the solutes. The solution was concentrated to about 60 mL by using a rotary evaporator. The concentrate was left overnight in a refrigerator. As a result, a white solid precipitated, which was filtered and collected. The weight was measured at 3.15 g after drying overnight in a reduced pressure at 40° C. Anaylsis with high speed liquid chromatography of the solid revealed almost pure inosine.

### Test Example 1

Test of Velocity of Dissolution

Each of the following was added to separate containers containing 10 mL of water with stirring at room temperature: (a) a mixture (0.53 g) of 1.2 mmol of inosine and 1.2 mmol of L-arginine, (b) 1.2 mmol (0.53 g) of the inosine. L-arginine salt obtained in Example 1, and (3) 1.2 mmol (0.32 g) of inosine. The time period for complete dissolution was measured for each. The equimolar mixture of inosine and L-arginine required 50 seconds for complete dissolution. On the other hand, inosine. L-arginine salt dissolved immediately. Furthermore, the inosine alone did not completely dissolve. From this, the superior dissolution properties of inosine. L-arginine salt were confirmed.

### Example 2

Plant Growth Promoting Effect

Lawn seedlings (Western lawn: bent grass) were raised and divided into two groups each containing 30 plants. Hydroponics were performed as follows. Both the control group and the inosine. L-arginine salt group were cultured using HYPONEX, diluted by a facter of 2,000. The HYPONEX diluted by a facter of 2000 alone was used as culture fluid for the control group. Inosine- L-arginine salt was added to the culture fluid to an inosine concentration of 20 ppm.

After a month of culturing, the average plant length, the average root length, the number of leaves for 30 plants, the number of branching of a stem for 30 plants, the weight for 30 plants, and the dried weight for 30 plants were measured for each group. The results are presented in Table 1. From the Table, the effectiveness of inosine. L-arginine salt was confirmed.

TABLE 1

| | Control | inosine · L-arginine salt |
|---|---|---|
| | group | group |
| Average plant length (cm) | 5.5 | 6.0 |
| Average root length (cm) | 4.5 | 5.0 |
| Number of leaves per 30 plants | 83 | 102 |
| Number of branching per 30 | 31 | 37 |
| plants | | |
| Weight (g) per 30 plants | 0.09 | 0.13 |
| Dried weight (g) per 30 plants | 0.0314 | 0.0434 |

### Industrial Applicability

According to the present invention, inosine. L-arginine salt is described which can be separated as a solid, is easily soluble in water, and is useful as a cell activator or plant growth promoter. Also described is a cell activator or a plant growth promoter containing inosine. L-arginine salt as an active ingredient. And, such cell activator or plant growth promoter is provided in the form of a preparation of an aqueous solution of inosine and L-arginine.

While the invention has been described in detail with reference to preferred embodiments thereof, it will be apparent to one skilled in the art that various changes can be made, and equivalents employed, without departing from the scope of the invention. Each of the aforementioned documents, including the foreign priority document JP 2001-297011, is incorporated by reference herein in its entirety.

We claim:

1. An inosine. L-arginine salt.

2. The inosine. L-arginine salt of claim 1, wherein inosine and L-arginine are present in substantially equimolar amounts

3. The inosine. L-arginine salt of claim 1 produced by the process of
a) dissolving in water inosine and L-arginine in substantially equimolar amounts,
b) drying the dissolution product.

4. The inosine. L-arginine salt of claim 3, wherein the process includes the additional step of adding the product of step (a) to anhydrous ethanol prior to step (b).

5. A composition comprising the inosine. L-arginine salt of claim 1.

6. A composition comprising an aqueous solution of inosine and L-arginine.

7. The composition of claim 6, wherein said inosine and said L-arginine are present in substantially equimolar amounts.

8. A method of promoting the growth of a plant comprising treating said plant with an inosine. L-arginine salt.

9. A method of promoting the growth of a plant comprising treating said plant with a composition comprising an aqueous solution of inosine and L-arginine.

10. The method of claim 9, wherein said inosine and said L-arginine are present in substantially equimolar amounts.

11. A method of activating a cell comprising treating the cell with an inosine. L-arginine salt.

12. A method of activating a cell comprising treating the cell with a composition comprising an aqueous solution of inosine and L-arginine.

13. The method of claim 12, wherein said inosine and said L-arginine are present in substantially equimolar amounts.

14. A method of making an inosine. L-arginine salt comprising
a) dissolving in water inosine and L-arginine in substantially equimolar amounts; and
b) adding the product of step (a) to anhydrous ethanol; and
c) drying the product of step (b) to obtain inosine. L-arginine salt.
